(19) United States
(12) Patent Application Publication
(10) Pub. No.: US 2021/0015415 A1
(43) Pub. Date: Jan. 21, 2021

(54) METHODS AND SYSTEMS FOR MONITORING USER WELL-BEING

(71) Applicant: Somatix, Inc., New York, NY (US)

(72) Inventors: Eran Ofir, Bazra (IL); Uri Schatzberg, Kiryat-Ono (IL)

(21) Appl. No.: 16/897,209

(22) Filed: Jun. 9, 2020

Related U.S. Application Data
(63) Continuation of application No. PCT/US2018/065833, filed on Dec. 14, 2018.
(60) Provisional application No. 62/599,567, filed on Dec. 15, 2017.

(51) Int. Cl. A61B 5/16; G06F 3/01; A61B 5/00; A61B 5/11; G16H 50/70; G16H 50/30; G16H 10/20; G16H 50/20; G16H 40/67

(52) U.S. Cl. CPC A61B 5/165; G06F 3/017; A61B 5/6801; A61B 5/1118; A61B 5/1117; A61B 5/4809; G16H 40/67; A61B 5/7275; G16H 50/70; G16H 50/30; G16H 10/20; G16H 50/20; A61B 5/7264

(57) ABSTRACT

Methods and systems are provided herein for monitoring a user's well-being. The methods may comprise collecting one or more sensor data, analyzing at least a subset of the collected sensor data, extracting features from the collected and/or analyzed sensor data, and determining one or more of a physical score, a psychological score and a total score. The methods may further comprise determining a user's well-being based on one or more of the scores.

### CROSS-REFERENCE

This application is a continuation application of International Application No. PCT/US2018/065833, filed Dec. 14, 2018, which claims the benefit of U.S. Provisional Patent Application No. 62/599,567, filed Dec. 15, 2017, which is entirely incorporated herein by reference.

### BACKGROUND

Physical and/or psychological state of an individual can be important to his/her general well-being and may affect various aspects of that individual's life, for example, effective decision making. People who are aware of their physical and/or psychological well-being can be better equipped to realize their own abilities, cope with stresses of life, work or other social events, and contribute to communities. However, the physical and/or psychological state of an individual, especially signs or precursors to certain health or mental well-being conditions, may not always be apparent and easily captured in the early stages. It is often preferable to address and take appropriate measures in the early stages compared to later stages.

### SUMMARY

There is a need for methods and systems that can monitor and assess physical and/or psychological states, and predict certain at-risk physical and/or psychological states at early stages of development, thus allowing for the possibility of preventive measures and efforts. There are many instances where it may be desirable to ascertain an individual's well-being. Determination of a person's well-being may comprise assessment of his/her physical and/or psychological state. Conventionally, to make such a determination, a health care professional typically either interacts with the person or the person is subjected to a couple of tests, in order to monitor the person's physical or psychological state. However, such determination may be subjective and thus inaccurate, as different health care professionals may reach different conclusions given the same test results. Therefore, accurate and reliable methods and systems for monitoring a user's well-being are needed.

An aspect of the present disclosure provides a computer-implemented method for determining a user's well-being, the method comprising: receiving data from a plurality of sources; analyzing the data received from the plurality of sources to detect gestures and events associated with the user; extracting a plurality of features from the data received from the plurality of sources and the analyzed data corresponding to the detected gestures and events; selecting one or more subsets of features from the plurality of extracted features; and using at least partially the selected one or more subsets of features to determine (i) a physical state of the user, (ii) a psychological state of the user, or (iii) physical and psychological states of the user, to thereby determine the user's well-being.

In some embodiments, the one or more subsets of features comprise a first subset of features and a second subset of features. In some embodiments, the method further comprises using at least partially the first subset of features and the second subset of features to determine the physical state of the user and the psychological state of the user respectively. In some embodiments, the first subset of features and the second subset of feature comprise common features. In some embodiments, the method further comprises using at least partially the common features to determine the physical and psychological state of the user. In some embodiments, the method further comprises adjusting the one or more subsets of features if an accuracy of the determination is lower than a predetermined threshold. In some embodiments, the accuracy is determined based on the user's feedback regarding the physical state, the psychological state, or the physical and psychological state of the user. In some embodiments, the adjusting is performed by adding, deleting, or substituting one or more features in the one or more subsets of features. In some embodiments, the adjusting is performed substantially in real-time. In some embodiments, the method further comprises determining (1) a physical score based on the physical state of the user, (2) a psychological score based on the psychological state of the user, and/or (3) a total score based on the physical and psychological states of the use. In some embodiments, the method further comprises sending queries regarding the determined physical state, the psychological state, and/or the physical and psychological states to the user; receiving responses to the queries from the user; and adjusting the physical score, the psychological score, and/or the total score based on the responses. In some embodiments, the method further comprises monitoring at least one of the physical and psychological states of the user as the gestures and events associated with the user are occurring. In some embodiments, the method further comprises determining a trend of at least one of the physical and psychological states of the user based on the monitoring. In some embodiments, the method further comprises predicting at least one of a future physical state or a psychological state of the user based on the trend. In some embodiments, the method further comprises determining different degrees of a given physical state or psychological state. In some embodiments, the method further comprises distinguishing between different types of physical and psychological states. In some embodiments, the plurality of sources comprises a wearable device and a mobile device associated with the user. In some embodiments, the data comprises sensor data collected using a plurality of sensors on the wearable device or mobile device. In some embodiments, the gestures comprise different types of gestures performed by an upper extremity of the user. In some embodiments, the events comprise (i) different types of activities and (ii) occurrences of low activity or inactivity. In some embodiments, the events comprise walking, drinking, taking medication falling, eating, and/or sleeping. In some embodiments, the plurality of features are processed using at least a machine learning algorithm or a statistical model. In some embodiments, the physical state comprises a likelihood that the user is physically experiencing conditions associated with the physical state, and the psychological state comprises a likelihood that the user is mentally or emotionally experiencing conditions associated with the psychological state. In some embodiments, the method further comprises comparing the likelihood(s) to one or more thresholds; and generating one or more alerts to the user or another entity, depending on whether the likelihood(s) are less than, equal, or greater than the one or more thresholds.

Another aspect of the present disclosure provides a computer-implemented method for determining a user's well-being, the method comprising: receiving data from a plurality of sources; analyzing the data received from the plurality of sources to detect gestures and events associated with the user; extracting a plurality of features from the data received from the plurality of sources and the analyzed data corresponding to the detected gestures and events; and processing at least a subset of the plurality of features to determine (1) individual physical and psychological states of the user, and (2) comparisons between the physical and psychological states influencing the user's well-being.

In some embodiments, the plurality of sources comprises a wearable device and a mobile device associated with the user. In some embodiments, the wearable device and/or the mobile device is connected to the internet. In some embodiments, the data comprises sensor data collected using a plurality of sensors on the wearable device or mobile device. In some embodiments, the gestures comprise different types of gestures performed by an upper extremity of the user. In some embodiments, the events comprise (i) different types of activities and (ii) occurrences of low activity or inactivity. In some embodiments, the events comprise walking, drinking, taking medication falling, eating, and/or sleeping. In some embodiments, the events comprise voluntary events and involuntary events associated with the user. In some embodiments, the comparisons between the physical and psychological states are used to more accurately predict the user's well-being. In some embodiments, the method further comprises determining (i) a physical score based on the physical state of the user, and (ii) a psychological score based on the psychological state of the user. In some embodiments, the method further comprises calculating a total well-being score of the user by aggregating the physical and psychological scores. In some embodiments, the plurality of features are processed using at least a machine learning algorithm or a statistical model. In some embodiments, a common subset of the features is processed to determine both the physical and psychological states of the user. In some embodiments, different subsets of the features are processed to individually determine the physical and psychological states of the user. In some embodiments, the physical state comprises a likelihood that the user is physically experiencing conditions associated with the physical state, and the psychological state comprises a likelihood that the user is mentally or emotionally experiencing conditions associated with the psychological state. In some embodiments, the method further comprises comparing the likelihood(s) to one or more thresholds; and generating one or more alerts to the user or another entity, depending on whether the likelihood(s) are less than, equal, or greater than the one or more thresholds. In some embodiments, the one or more thresholds comprise at least one threshold that is specific to or predetermined for the user. In some embodiments, the one or more thresholds comprise at least one threshold that is applicable across a reference group of users.

It shall be understood that different aspects of the disclosure can be appreciated individually, collectively, or in combination with each other. Various aspects of the disclosure described herein may be applied to any of the particular applications set forth below or for any other types of energy monitoring systems and methods.

Other objects and features of the present disclosure will become apparent by a review of the specification, claims, and appended figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flowchart of an example method for monitoring user well-being, in accordance with some embodiments;

FIG. 2 illustrates an example system for monitoring user well-being, in accordance with some embodiments;

FIG. 3 illustrates an example method for acquiring data from a subject, in accordance with some embodiments;

FIG. 4 illustrates sample components of an example system for monitoring user well-being, in accordance with some embodiments;

FIG. 5 depicts example components of an example subsystem for sensor data acquisition, in accordance with some embodiments;

FIG. 6A illustrates an example method for determining locations of a user, in accordance with some embodiments;

FIGS. 6B and 6C show signal strength distributions at two different locations determined using the method of FIG. 6A, in accordance with some embodiments;

FIGS. 7A and 7B show example methods for determining user well-being using sensor data, in accordance with some embodiments;

FIG. 8 shows example data collected during a given time period from a user, in accordance with some embodiments; and

FIG. 9 shows a computer system that is programmed or otherwise configured to implement any of the methods provided herein.

### DETAILED DESCRIPTION

Reference will now be made in detail to some exemplary embodiments of the disclosure, examples of which are illustrated in the accompanying drawings. Wherever possible, the same reference numbers will be used throughout the drawings and disclosure to refer to the same or like parts.

Methods and systems of the present disclosure may utilize sensors to collect data from a user. The sensor data may be used to detect gestures and/or events associated with the user. The events may comprise voluntary events (such as running, walking, eating, drinking) and involuntary events (such as falling, tripping, slipping). The collected sensor data and detected gesture and events may be utilized to determine physical and/or psychological state of the user. To make the determination, the methods and systems may further comprise generating a physical score, a psychological score, and/or a total health score using the sensor data and/or detected gestures/events. In some cases, when the physical and/or psychological state of a user degrades to a certain point (such as a pre-defined threshold level), the methods and systems may further comprise sending notifications to the user and/or a third party to report the determined physical and/or psychological state. The notifications may be messages such as text messages, audio messages, video messages, picture messages, or any types of multimedia messages, or combinations thereof. The notification may be a report generated based on the determined physical and/or psychological state. The notification may be sent to one or more receiving parties. The receiving parties may be a person, an entity, or a server. Non-limiting examples of the receiving parties include spouses, friends, parents, caregivers, family members, relatives, insurance company, living assistants, nurses, physicians, employer, coworkers, emergency response team, and a server.

An aspect of the present disclosure provides methods for monitoring user well-being. The methods may be computer-implemented methods. The methods may comprise receiving data from a plurality of sources. The sources may be associated with the user. The sources may comprise sensors, wearable devices, mobile devices, user devices or combinations thereof. The data received from the plurality of sources may be raw data. Thus, the methods may further comprise analyzing at least a subset of the data received from the plurality of sources to detect gestures and/or events associated with the user. The gestures may be any movement (voluntary or involuntary) of at least a part of the body made by the user. For example, the gestures may comprise different types of gestures performed by an upper and/or lower extremity of the user. The events may be any voluntary or involuntary events. The events may comprise different types of activities and/or occurrences of low activity or inactivity. Non-limiting examples of the gestures/events may include all activities of daily living (ADL's), smoking, eating, drinking, taking medication, falling, tripping, slipping, brushing teeth, washing hands, bathing, showering, walking (e.g., number of steps, step length, distance of waking, walking speed), gait changes, sleeping (e.g., sleeping time, quality, number of wake-ups during sleeping), toileting, active time during the day, indoor/outdoor activities, indoor/outdoor locations, indoor/outdoor locations the user goes frequently (or points-of-interests (POI's)), number of times the user goes to the POI'S, number of times the user spends in bed, sofa, chair, a given POI, and/or any given location during a certain time period, transferring from/to a given location, bed time, time/frequency of phone speaking, number and duration of phone calls, time/duration of a day the user spent at an indoor/outdoor location, patterns of speech, patterns of non-verbal sounds such as bodily sounds from respiration, digestion, and breathing, or combinations thereof.

As will be appreciated, the sensor data receipt/collection or acquisition and gesture/event detection or determination can be performed simultaneously, sequentially or alternately. Upon receipt of the sensor data and/or detection of the gestures/events associated with the user, at least a subset of the sensor data and the detected gestures/events may be used for extracting features which may be used for determining the user's well-being. The determination may comprise processing at least a subset of the plurality of features to determine (1) individual physical and psychological states of the user, and (2) comparisons between the physical and psychological states influencing the user's well-being. In some cases, the physical state comprises a likelihood that the user is physically experiencing conditions associated with the physical state. In some cases, the psychological state comprises a likelihood that the user is mentally and/or emotionally experiencing conditions associated with the psychological state. The likelihood may be compared to one or more pre-defined thresholds. In some cases, one or more alerts or notifications may be generated, depending on whether the likelihood(s) are less than, equal to or greater than the thresholds. The thresholds may comprise a threshold that is specific to or predetermined for the user. The thresholds may comprise a threshold that is applicable across a reference group of users. For example, the threshold may comprise a threshold calculated using a reference group of users that have the same age, sex, race, employment, and/or education as the user.

In some cases, the methods may further comprise monitoring the physical and/or psychological states of the user substantially in real-time. In some cases, the monitoring may comprise determining the physical and/or psychological states of the user multiple times within a pre-defined time period. For example, the physical and/or psychological states of the user may be determined at least 2, 3, 4, 5, 6, 7, 8, 9, 10 times a day or 2, 3, 4, 5, 6, 7, 8, 9, 10 times or more per day for consecutive 2, 3, 4, 5, 6, 7, 8, 9, 10 days or more. In some cases, as changes in the physical and/or psychological states are detected, the one or more generated scores (e.g., the physical score, the psychological score, the total score) may be updated. The scores may be updated dynamically. The scores may be updated substantially in real-time.

Alternatively or additionally, the determination may comprise generating one or more scores using at least a subset of the plurality of features. The one or more scores may comprise a physical score, a psychological score and a total health or well-being score. In some instances, the total well-being score may be generated by aggregating the physical and psychological scores. Such aggregation may be performed according to the comparisons between the physical and psychological states. Upon generation of the one or more scores, the individual physical and psychological states of the user, and/or comparisons between the physical and psychological states influencing the user's well-being may be determined based on the scores.

The individual physical and psychological states may be determined using different subsets of the features. The different subsets of the features used to determine the physical and psychological states may comprise common features. The common features may be utilized to determine comparisons between the physical states and psychological states of the user.

Comparisons between the physical and psychological states may comprise different degrees of comparisons. For example, poor psychological state (or mental health) is a risk factor for chronic physical conditions. In another example, people with serious mental health conditions may be at high risk of having a poor physical state or experiencing deteriorating physical conditions. Similarly, poor physical state or conditions may lead to an increased risk of developing mental health problems. Comparisons between the physical and psychological states may be determined based on a comparison coefficient calculated using one or more of the extracted features. The comparison coefficient may be compared to a pre-defined reference value (or threshold). The reference value may be determined using a reference group such as a reference group selected based on age, sex, race, income, employment, location, medical history or any other factors.

In some cases, one or more of the physical states may be highly associated with one or more of the psychological states. The comparisons between the physical and psychological states may be used to more accurately predict the user's well-being.

In some instances, methods of the present disclosure may comprise receiving data from a plurality of sources. The data may comprise raw data. The received data may be collected, stored, and/or analyzed. The data may be transformed into user information data. The data transformation may comprise amplifying, filtering, multiplexing, digitizing, reducing or eliminating noise, converting signal types (e.g., converting digital signals into radio frequency signals), and/or other processing methods specific to the type of data received from the sources.

In some cases, the methods may further comprise analyzing at least a subset of the received data to detect gestures and/or events associated with a user. As described above and elsewhere herein, the gestures may be any movement (voluntary or involuntary) of at least a part of the body of the user. For example, the gestures may comprise different types of gestures performed by an upper and/or lower extremity of the user. The events may be any voluntary or involuntary events. Examples of the gestures and events have been described above and elsewhere herein. At least a subset of the received data and detected gestures/events may be used for extracting features. A plurality of features may be extracted. Once the features are extracted, the methods may further comprise selecting one or more subsets (e.g., 2, 3, 4, 5, 6, 7, 8, 9, 10 subsets or more) of the generated features. The selected one or more subsets of the features may then be used for determining one or more of (i) a physical state (ii) a psychological state and (iii) a physical and psychological state of the user, thereby identifying general well-being of the user. The determination may comprise determining different degrees or stages of a given physical, psychological and/or physical and psychological states. The determination may comprise distinguishing different types of physical, psychological and/or physical and psychological states.

In some cases, the determination may use a portion of selected one or more subsets of the features. In some examples, the methods may comprise selecting, from a plurality of features, a first subset, a second subset and a third subset of the features. The first, second and third subsets of the features may or may not comprise common features. The first subset, the second subset and the third subset of the features may be utilized to determine the physical state, the psychological state and the physical and psychological state of the user, respectively. In some cases, the methods may further comprise generating one or more scores which may be indicative of a user's well-being. For example, a physical score may be generated for determining a physical state of the user. Similarly, a psychological score and/or a total well-being score may be generated to determine the user's psychological state and/or a physical and psychological state.

The one or more scores may be generated using at least partially the extracted features. The features used to generate the physical score, psychological score and total score may or may not be the same. In some cases, each of the physical score, psychological score and total score may be generated using a different subset of the features. Such different subsets of the features may or may not comprise common features. As an example, features that may be utilized for determining a physical score may comprise smoking, sleeping (e.g., duration and/or quality of the sleep), active time in a day, steps (including number and length of steps), eating, drinking, taking medication, falling, gait changes, number of times a day a user visits POI, time a user spends in bed, sofa, chair or any given location, transferring to or from a given location, lying in bed, speaking detection (using microphone), total time in a day a user is speaking, number and duration of phone calls, time in a day a user spends on mobile devices such as smart phone, time of a day a user spends outdoors, blood pressure, heart rate, galvanic skin response (GSR), oxygen saturation, and/or explicit feedback from user answers.

In another example, a psychological score may be determined based on features which may comprise eating, drinking, taking medication, brushing teeth, walking (e.g., number of steps, time and duration), sleeping (e.g., duration and/or quality of the sleep), showering, washing hands, active time in a day, number of times a day a user visits POI, speaking detection (using microphone), total time in a day a user is speaking (detected using microphone), number and duration of phone calls, time and duration in a day a user is using smart phone, time an duration in a day a user spends outside, blood pressure, heart rate, GSR, transferring to or from a given location, lying in bed, and/or explicit feedback from user answers.

In another example, a physical and psychological score may be determined using features that may be associated to both the physical and psychological state of a user, for example, sensor data, gestures and/or events that may be related to both the physical and psychological state of a user. Non-limiting examples of such features may comprise location of a user (e.g., indoor, outdoor), environmental data (e.g., weather, temperature, pressure), time, duration and/or quality of sleep, number of steps, time and duration of phone calls, and/or active time in a day.

The extracted features may be transformed and/or processed into physical, psychological, and/or physical and psychological states using various techniques such as machine learning algorithms or statistical models. The extracted features may be transformed and/or processed into physical, psychological, and/or physical and psychological scores using various techniques such as machine learning algorithms or statistical models.

Machine learning algorithms that may be used in the present disclosure may comprise supervised (or predictive) learning, semi-supervised learning, active learning, unsupervised machine learning, or reinforcement learning. Non-limiting examples of machine learning algorithms may comprise support vector machines (SVM), linear, logistics, tress, random forest, xgboost, neural networks, deep neural networks, boosting techniques, bootstrapping techniques, ensemble techniques, or combinations thereof.

Prior to applying the machine learning algorithm(s) on the data and/or the detected gestures/events, some or all of the data, gestures, and/or events may be preprocessed or transformed to make it meaningful and appropriate for the machine learning algorithm(s). For example, a machine learning algorithm may require “attributes” of the received data or detected gestures/events, to be numerical or categorical. It may be possible to transform numerical data to categorical and vice versa. Categorical-to-numerical methods may comprise scalarization, in which different possible categorical attributes are given different numerical labels, for example, “fast heart rate,” “high skin temperature,” and “high blood pressure” may be labeled as the vectors [1, 0, 0], [0, 1, 0], and [0, 0, 1], respectively. Further, numerical data can be made categorical by transformations such as binning. The bins may be user-specified, or can be generated optimally from the data.

In some cases, the selected subsets of the features may be adjusted if an accuracy of the determination is lower than a pre-determined threshold. The pre-determined threshold may correspond to a known physical, psychological and/or physical and psychological state. Each known physical, psychological and/or physical and psychological state may be associated with user information including a set of specific features (optionally with known values) which may be used as standard user information for that particular state. In some cases, the standard user information for a given physical, psychological and/or physical and psychological state may be obtained by exposing a control group or reference group of users to one or more controlled environment (e.g., with preselected activities or interactions with preselected environmental conditions), monitoring users' responses, collecting data (some or all types of sensor data described herein), and detecting gestures/events for such controlled environment. The user information obtained under such controlled environment may be representative and may be used for generating one or more pre-determined thresholds for a given physical, psychological and/or physical and psychological state.

Adjusting selected subsets of the features may be performed by adding, deleting, or substituting one or more features in the selected subsets of features. The adjusting may be performed substantially in real-time. For example, once it is determined that an accuracy of determined state is lower than a pre-defined value, there may be little or no delay for the system to make adjustment to the selected subset(s) of the features that is used to determine that particular state.

In some cases, upon determination of a physical, psychological and/or physical and psychological state of the user, one or more queries regarding the determined state(s) may be sent to the user and/or a third party. The queries may comprise a query requesting user's feedback, comments or confirmation of the determined state. The queries may comprise a query requesting the user to provide additional information regarding the determined state. Responses or answers to the queries may be received from the user. The responses may comprise no response after a certain time period (e.g., after 10 minutes (min), 15 min, 30 min, 45 min, 1 hours (hr), 2 hr, 3 hr, 4 hr, 5 hr, or more). The responses may be used for adjusting the generated physical, psychological and/or physical and psychological scores. In some cases, depending upon the received responses from the user, further actions may be taken. For example, if the user confirms that he/she is experiencing conditions associated with an adverse physical, psychological and/or physical and psychological state, notifications or alerts may be sent to a third party such as health provider, hospital, emergency medical responders, family members, friends, and/or relatives.

In some cases, the methods may further comprise monitoring a physical, psychological and/or physical and psychological state of the user. The monitoring may occur during a pre-defined time period (e.g. ranging from hours or days to months). Based on the monitoring, a trend of the physical, psychological and/or physical and psychological state of the user may be identified. Alternatively or additionally, a future physical, psychological and/or physical and psychological state may be predicted based on the monitoring and/or the identified trend.

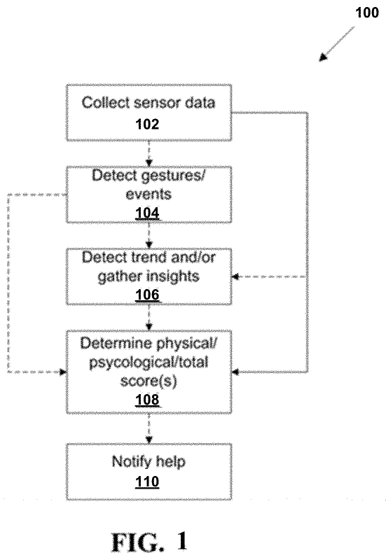

FIG. 1 shows an example method 100 for detecting a user's well-being, in accordance with some embodiments. First, a plurality of sensor data may be collected 102 from a variety of sources. The sources may comprise sensors and/or devices (including such as user devices, mobile devices, wearable devices etc.). The received data may comprise raw data. The received data may be analyzed or processed for detecting gestures and/or events 104. The gestures and/or events may be voluntary or involuntary. The gestures and/or events may be associated with the user. Optionally, a trend of data may be detected 106. In some cases, one or more features may be extracted by processing the received data and/or the detected gestures/events to gather insights and further information from the received data 106. Next, at least a subset of the extracted data may be used for determining one or more scores 108 related to a physical, psychological and/or physical and psychological state of the user. As described above and elsewhere herein, the same or different subsets of the features may be utilized to generate a physical score, a psychological score and a total score. The generated scores may then be used for determining a physical state, a psychological state and/or a physical and psychological state of the user, thereby determining the user's well-being. In some cases, notification 110 concerning the determined user state may be sent to the user or another party (may be a person or an entity). The notification may comprise a notification for help.

Also provided herein are systems for monitoring user well-being. The systems may comprise a memory and one or more processors. The memory may be used for storing a set of software instructions. The one or more processors may be configured to execute the set of software instructions. Upon execution of the instructions, one or more methods of the present disclosure may be implemented. The systems may further comprise one or more additional components. In some cases, the components may comprise one or more devices including sensors, user devices, mobile devices, and/or wearable devices, one or more engines (e.g., gesture/event detection engine, gesture/event analysis engine, feature extraction (or analysis) engine, feature selection engine, score determination engine etc.), one or more servers, one or more databases and any other components that may be suitable for implementing methods of the present disclosure. Various components of the systems may be operatively coupled or in communication with one another. For example, the one or more servers may be in communication with some or all of the devices or engines. In some cases, the one or more devices may be integrated into a single device which may perform multiple functions. In some cases, the one or more engines may be combined or integrated into a single engine.

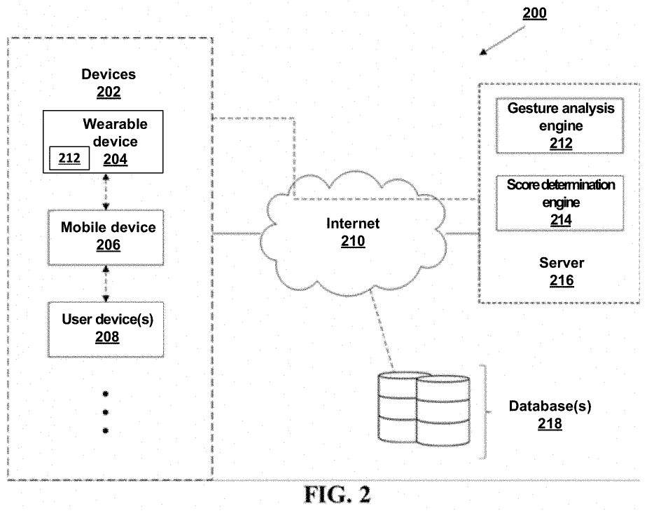

FIG. 2 illustrates an example system for monitoring user well-being, in accordance with some embodiments. As shown in the figure, an example system 200 may comprise one or more devices 202 such as a wearable device 204, a mobile device 206 and a user device 208, one or more engines such as a gesture analysis engine 212, a feature extraction engine (not shown) and a score determination engine 214, a server 216 and one or more databases 218. Some or all of the components may be operatively connected to one another via network 210 or any type of communication links that allows transmission of data from one component to another.

The one or more devices may comprise sensors. The sensors can be any device, module, unit, or subsystem that may be configured to detect a signal or acquire information. Non-limiting examples of sensors include inertial sensors (e.g., accelerometer, gyroscopes, gravity detection sensors which may form inertial measurement units (IMUs)), location sensors (e.g., global positioning system (GPS) sensors, mobile device transmitters enabling location triangulation), heart rate monitors, temperature sensors (e.g., external temperature sensors, skin temperature sensors), environmental sensors configured to detect parameters associated with an environment surrounding the user (e.g., temperature, humidity, brightness), capacitive touch sensors, GSR sensors, vision sensors (e.g., imaging devices capable of detecting visible, infrared, or ultraviolet light, cameras), thermal imaging sensors, location sensors, proximity of range sensors (e.g., ultrasound sensors, light detection and ranging (LIDAR), time-of-flight or depth cameras), altitude sensors, attitude sensors (e.g., compasses), pressure sensors (e.g., barometers), humidity sensors, vibration sensors, audio sensors (e.g., microphones), field sensors (e.g., magnetometers, electromagnetic sensors, radio sensors), photoplethysmogram (PPG) sensors, blood pressure sensors, liquid detectors, Wi-Fi, Bluetooth, cellular network signal strength detectors, ambient light sensors, ultraviolet (UV) sensors, oxygen saturation sensors, or combinations thereof. The sensors may be comprised in or located on one or more of the wearable devices, mobile devices, and user devices. In some cases, a sensor may be placed inside the body of the user.

The user device 208 may be a computing device configured to perform one or more operations consistent with the disclosed embodiments. Non-limiting examples of user devices may include, mobile devices, smartphones/cellphones, tablets, personal digital assistants (PDAs), laptop or notebook computers, desktop computers, media content players, television sets, video gaming station/system, virtual reality systems, augmented reality systems, microphones, or any electronic device capable of analyzing, receiving, providing or displaying certain types of behavioral data (e.g., smoking data) to a user. The user device may be a handheld object. The user device may be portable. The user device may be carried by a human user. In some cases, the user device may be located remotely from a human user, and the user can control the user device using wireless and/or wired communications.

The use device may comprise one or more processors that are capable of executing non-transitory computer readable media that may provide instructions for one or more operations consistent with the disclosed embodiments. The user device may include one or more memory storage devices comprising non-transitory computer readable media including code, logic, or instructions for performing the one or more operations. The user device may include software applications that allow the user device to communicate with and transfer data among various components of a system (e.g., sensors, wearable devices, gesture/events analysis engine, score determination engine, and serve). For example, the user device may include software applications that allow the user device to communicate with and transfer data between wearable device 204, mobile device 206, gesture detection/analysis engine 212, score determination engine 214, feature extraction (or analysis) engine, and/or database(s) 218. The user device may include a communication unit, which may permit the communications with one or more other components comprised in the system 200. In some instances, the communication unit may include a single communication module, or multiple communication modules. In some instances, the user device may be capable of interacting with one or more components in the system using a single communication link or multiple different types of communication links.

The user device 208 may include a display. The display may be a screen. The display may or may not be a touchscreen. The display may be a light-emitting diode (LED) screen, OLED screen, liquid crystal display (LCD) screen, plasma screen, or any other type of screen. The display may be configured to show a user interface (UI) or a graphical user interface (GUI) rendered through an application (e.g., via an application programming interface (API) executed on the user device). The GUI may show graphical elements that permit a user to monitor collected sensor data, generated scores, view a notification or report regarding his/her physical/psychological state, view queries prompted by health care provider regarding determined physical/psychological state. The user device may also be configured to display webpages and/or websites on the Internet. One or more of the webpages/websites may be hosted by a server 216 and/or rendered by the one or more engines comprised in the system 200.

In some embodiments, the one or more engines may comprise a rule engine. The rule engine may be configured to determine a set of rules associated with a physical state, a psychological state, and/or a physical and psychological state. The set of rules may or may not be personalized. The set of rules may be stored in database(s) (e.g., rule repository). The set of rules may comprise rules that may be used for transforming features into scores. Each feature may be individually evaluated to generate a score. Scores of features associated with a given physical state, a psychological state, and/or a physical and psychological state may be aggregated to generate an aggregated score. The aggregated score may be compared to a predetermined threshold(s). A physical state, a psychological state, and/or a physical and psychological state may be determined, depending upon, whether the aggregated score is lower than, equal to, or higher than the predetermined threshold(s). As an example, if the feature comprises number of steps a day and duration of sleep, a set of rules may comprise: (1) if a user walks more than 100 steps, then add 2 to his score; (2) if a user walks more than 1,000 steps, then add 20 to his score; (3) if a user walks more than 10,000 steps, then add 40 to his score; (4) if a user sleeps more than 5 hours a day, then add 5 to his score; (5) if a user sleeps more than 7 hours a day, then add 15 to his score; and (6) if a user sleeps more than 9 hours a day, then add 20 to his score. Assuming a user being monitored walks 200 steps a day and sleeps 8 hours a day, then a total of 17 may be added to his score corresponding to the feature.

In some cases, the set of rules may comprise a pattern indicative of one or more gestures/events associated with a given physical, psychological and/or physical and psychological state. The pattern may or may not be the same across different users. In some embodiments, the pattern may be obtained using an artificial intelligence algorithm such as an unsupervised machine learning method. The pattern may be user-specific. In some cases, a pattern associated with a user may be obtained by training a model over datasets related to the user. In some cases, the model may be obtained automatically without user input. For instance, the dataset related to the user may be collected from devices worn or carried by the user and/or data that are input by the user. The model may be time-based. In some cases, the model may be updated and refined in real-time as further user data is collected and used for training the model. Alternatively or additionally, the model may be trained during an initialization phase until one or more user attributes are identified. For instance, device data during the initialization phase may be collected to identify attributes such as a walking pattern, sleeping pattern, drinking/eating pattern, a POI or any given geolocation and a frequency of a user visiting the POI(s) or certain geolocations and the like. The identified user attributes may then be factored into determining abnormal events. In some cases, the model may be trained over datasets aggregated from a plurality of devices worn or carried by a plurality of users. The plurality of users may be control or reference groups. The plurality of users may share certain user attributes such as geographical, age, gender, employment, life style, wellness (e.g., smoking, diet, cognitive psychology, diseases, emotional, mental and/or physical wellness) and various others.

A user may navigate within the GUI through the application. For example, the user may select a link by directly touching the screen (e.g., touchscreen). The user may touch any portion of the screen by touching a point on the screen. Alternatively, the user may select a portion of an image with aid of a user interactive device (e.g., mouse, joystick, keyboard, trackball, touchpad, button, verbal commands, gesture-recognition, attitude sensor, thermal sensor, touch-capacitive sensors, or any other device). A touchscreen may be configured to detect location of the user's touch, length of touch, pressure of touch, and/or touch motion, whereby each of the aforementioned manner of touch may be indicative of a specific input command from the user.

The application executed on the user device may deliver a message or notification upon determination of a physical and/or a psychological state of the user. Alternatively or additionally, the application executed on the user device may generate one or more scores that are indicative of the user's physical/psychological state, or general well-being. The score may be displayed to the user within the GUI of the application.

The user device 208 may provide device status data to the one or more engines of the system 200. The device status data may comprise, for example, charging status of the device (e.g., connection to a charging station), connection to other devices (e.g., the wearable device), power on/off, battery usage and the like. The device status may be obtained from a component of the user device (e.g., circuitry) or sensors of the user device.

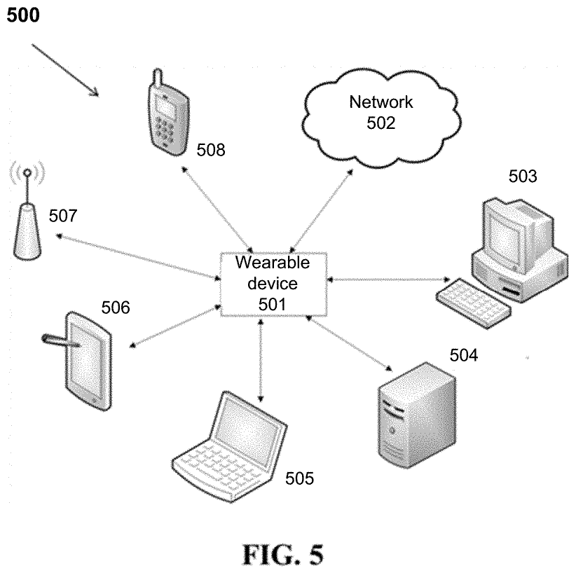

The wearable device 204 may include smartwatches, wristbands, finger rings, glasses, gloves, headgear (such as hats, helmets, virtual reality headsets, augmented reality headsets, head-mounted devices (HMD), headbands), pendants, armbands, leg bands, shoes, vests, motion sensing devices, etc. The wearable device may be configured to be worn on a part of a user's body (e.g., a smartwatch or wristband may be worn on the user's wrist). The wearable device may be in communication with other devices (503-508 in FIG. 5) and network 502.

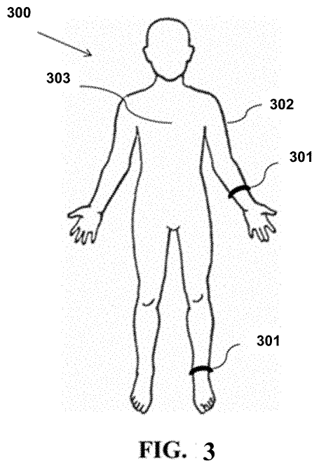

FIG. 3 shows an example method 300 for collecting sensor data from a user using one or more wearable devices 301. The wearable devices may be configured to be worn by the user on his/her upper and/or lower extremities. The wearable devices may comprise one or more types of sensors which may be configured to collect data inside 303 and outside 302 of the human body of the user. For example, the wearable device 301 may comprise sensors which may be configured to measure physiological data of the user such as blood pressure, heartbeat and heart rate, skin perspiration, skin temperature, oxygen saturation level, presence of cortisol in saliva etc. In some cases, the sensor data may be stored in a memory on the wearable device when the wearable device is not in operable communication with the user device and/or the server. In those instances, the sensor data may be transmitted from the wearable device to the user device when operable communication between the user device and the wearable device is re-established. Alternatively, the sensor data may be transmitted from the wearable device to the server when operable communication between the server and the wearable device is re-established.

In some cases, a wearable device may further include one or more devices capable of emitting a signal into an environment. For instance, the wearable device may include an emitter along an electromagnetic spectrum (e.g., visible light emitter, ultraviolet emitter, infrared emitter). The wearable device may include a laser or any other type of electromagnetic emitter. The wearable device may emit one or more vibrations, such as ultrasonic signals. The wearable device may emit audible sounds (e.g., from a speaker). The wearable device may emit wireless signals, such as radio signals or other types of signals.

In some cases, the one or more devices (such as the wearable device 204, the mobile device 206 and the user device 208) may be integrated into a single device. For example, the wearable device may be incorporated into the user device, or vice versa. Alternatively or additionally, the user device may be capable of performing one or more functions of the wearable device or the mobile device.

The one or more devices 202 may be operated by one or more users consistent with the disclosed embodiments. In some cases, a user may be associated with a unique user device and a unique wearable device. Alternatively, a user may be associated with a plurality of user devices and wearable devices.

The server 216 may be one or more server computers configured to perform one or more operations consistent with the disclosed embodiments. In some cases, the server may be implemented as a single computer, through which the one or more devices 202 may be able to communicate with the one or more engines and database 218 of the system. In some embodiments, the devices may communicate with the gesture analysis engine directly through the network. In some embodiments, the server may communicate on behalf of the devices with the gesture analysis engine or database through the network. In some embodiments, the server may embody the functionality of one or more of gesture analysis engines. In some embodiments, the one or more engines may be implemented inside and/or outside of the server. For example, the gesture analysis engine may be software and/or hardware components included with the server or remote from the server.

In some embodiments, the devices 202 may be directly connected to the server through a separate link (not shown in the figure). In certain embodiments, the server may be configured to operate as a front-end device configured to provide access to the one or more engines consistent with certain disclosed embodiments. The server may be configured to receive, collect and store data received from the one or more devices 202. The server may also be configured to store, search, retrieve, and/or analyze data and information (e.g., medical record/history, historical events, prior determination of physical, psychological state or general well-being, prior physical, psychological scores or total well-being scores) stored in one or more of the databases. The data may comprise a variety of sensor data collected from a plurality of sources associated with the user. The sensor data may be obtained using one or more sensors which may be comprised in or located on the one or more devices. The server 216 may also be configured to utilize the one or more engines to process and/or analyze the data. For example, the server may be configured to detect gestures and/or events associated with the user using the gesture analysis engine 212. In another example, the server may be configured to extract a plurality of features from at least a subset of the data and detected gestures/events using the feature extraction engine. At least a portion of the extracted features may then be used to determine one or more scores indicative of the user's physical, psychological state or general well-being, using the score determination engine 214.

A server may include a web server, an enterprise server, or any other type of computer server, and can be computer programmed to accept requests (e.g., HTTP, or other protocols that can initiate data transmission) from a computing device (e.g., user device, mobile device, wearable device etc.) and to serve the computing device with requested data. In addition, a server can be a broadcasting facility, such as free-to-air, cable, satellite, and other broadcasting facility, for distributing data. A server may also be a server in a data network (e.g., a cloud computing network).

A server may include known computing components, such as one or more processors, one or more memory devices storing software instructions executed by the processor(s), and data. A server can have one or more processors and at least one memory for storing program instructions. The processor(s) can be a single or multiple microprocessors, field programmable gate arrays (FPGAs), or digital signal processors (DSPs) capable of executing particular sets of instructions. Computer-readable instructions can be stored on a tangible non-transitory computer-readable medium, such as a flexible disk, a hard disk, a CD-ROM (compact disk-read only memory), and MO (magneto-optical), a DVD-ROM (digital versatile disk-read only memory), a DVD RAM (digital versatile disk-random access memory), or a semiconductor memory. Alternatively, the methods can be implemented in hardware components or combinations of hardware and software such as, for example, ASICs, special purpose computers, or general purpose computers.

While FIG. 2 illustrates the server as a single server, in some embodiments, multiple devices may implement the functionality associated with server.

Network 210 may be a network that is configured to provide communication between the various components illustrated in FIG. 2. The network may be implemented, in some embodiments, as one or more networks that connect devices and/or components in the network layout for allowing communication between them. For example, the one or more devices and engines of the system 200 may be in operable communication with one another over network 210. Direct communications may be provided between two or more of the above components. The direct communications may occur without requiring any intermediary device or network. Indirect communications may be provided between two or more of the above components. The indirect communications may occur with aid of one or more intermediary device or network. For instance, indirect communications may utilize a telecommunications network. Indirect communications may be performed with aid of one or more router, communication tower, satellite, or any other intermediary device or network. Examples of types of communications may include, but are not limited to: communications via the Internet, Local Area Networks (LANs), Wide Area Networks (WANs), Bluetooth, Near Field Communication (NFC) technologies, networks based on mobile data protocols such as General Packet Radio Services (GPRS), GSM, Enhanced Data GSM Environment (EDGE), 3G, 4G, or Long Term Evolution (LTE) protocols, Infra-Red (IR) communication technologies, and/or Wi-Fi, and may be wireless, wired, or a combination thereof. In some embodiments, the network may be implemented using cell and/or pager networks, satellite, licensed radio, or a combination of licensed and unlicensed radio. The network may be wireless, wired, or a combination thereof.

The devices and engines of the system 200 may be connected or interconnected to one or more databases 218. The databases may be one or more memory devices configured to store data. Additionally, the databases may also, in some embodiments, be implemented as a computer system with a storage device. In one aspect, the databases may be used by components of the network layout to perform one or more operations consistent with the disclosed embodiments.

The database(s) may comprise storage containing a variety of data sets consistent with disclosed embodiments. For example, the databases 218 may include, for example, raw data collected by and received from various sources including such as the one or more devices 202. In another example, the databases 218 may include a rule repository comprising rules associated with a given physical, psychological, and/or physical and psychological state. The rules may be predetermined based on data collected from a reference group of users. The rules may be customized based on user-specific data. The user-specific data may comprise data that are related to user's preferences, medical history, historical behavioral patterns, historical events, users' social interaction, statements or comments indicative of how the user is feeling at different points in time, etc. In some embodiments, the database(s) may include crowd-sourced data comprising comments and insights related to physical, psychological, and/or physical and psychological states of the user obtained from internet forums and social media websites or from comments and insights directly input by one or more other users. The Internet forums and social media websites may include personal and/or group blogs, Facebook™, Twitter™, etc.

In certain embodiments, one or more of the databases may be co-located with the server, may be co-located with one another on the network, or may be located separately from other devices (signified by the dashed line connecting the database(s) to the network). One of ordinary skill will recognize that the disclosed embodiments are not limited to the configuration and/or arrangement of the database(s).

The one or more databases may utilize any suitable database techniques. For instance, structured query language (SQL) or “NoSQL” database may be utilized for storing collected data, user information, detected gestures/events, rules, information of control/reference groups etc. The database of the present invention may be implemented using various standard data-structures, such as an array, hash, (linked) list, struct, structured text file (e.g., XML), table, JSON, NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present invention is implemented as a data-structure, the use of the database of the present invention may be integrated into another component such as any components of the present invention. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated. In some embodiments, the event detection system may construct the database in order to deliver the data to the users efficiently. For example, the event detection system may provide customized algorithms to extract, transform, and load the data. In some embodiments, the system may construct the databases using proprietary database architecture or data structures to provide an efficient database model that is especially adapted to large scale databases, is easily scalable, and has reduced memory requirements in comparison to using other data structures.

Any of the devices and the database may, in some embodiments, be implemented as a computer system. Additionally, while the network is shown in FIG. 2 as a “central” point for communications between components, the disclosed embodiments are not so limited. For example, one or more components of the network layout may be interconnected in a variety of ways, and may in some embodiments be directly connected to, co-located with, or remote from one another, as one of ordinary skill will appreciate. Additionally, while some disclosed embodiments may be implemented on the server, the disclosed embodiments are not so limited. For instance, in some embodiments, other devices (such as gesture analysis system(s) and/or database(s)) may be configured to perform one or more of the processes and functionalities consistent with the disclosed embodiments, including embodiments described with respect to the server.

Although particular computing devices are illustrated and networks described, it is to be appreciated and understood that other computing devices and networks can be utilized without departing from the spirit and scope of the embodiments described herein. In addition, one or more components of the network layout may be interconnected in a variety of ways, and may in some embodiments be directly connected to, co-located with, or remote from one another, as one of ordinary skill will appreciate.

The gesture analysis engine(s) may be implemented as one or more computers storing instructions that, when executed by processor(s), analyze input data from one or more of the devices 202 in order to detect gestures and events associated with the user. The gesture analysis engine(s) may also be configured to store, search, retrieve, and/or analyze data and information stored in one or more databases. In some embodiments, server 216 may be a computer in which the gesture analysis engine is implemented.

However, in some embodiments, the gesture analysis engine(s) 212 may be implemented remotely from server 216. For example, a user device may send a user input to server 216, and the server may connect to one or more gesture analysis engine(s) 212 over network 210 to retrieve, filter, and analyze data from one or more remotely located database(s) 218. In other embodiments, the gesture analysis engine(s) may represent software that, when executed by one or more processors, perform processes for analyzing data to detect gesture and events, and to provide information to the score determination engine(s) and/or feature extraction engine(s) for further data processing.

A server may access and execute the one or more engines (e.g., gesture analysis engine, score determination engine etc.) to perform one or more processes consistent with the disclosed embodiments. In certain configurations, the engines may be software stored in memory accessible by a server (e.g., in memory local to the server or remote memory accessible over a communication link, such as the network). Thus, in certain aspects, the engines may be implemented as one or more computers, as software stored on a memory device accessible by the server, or a combination thereof. For example, one gesture analysis engine may be a computer executing one or more gesture recognition techniques, and another gesture analysis engine may be software that, when executed by a server, performs one or more gesture recognition techniques.

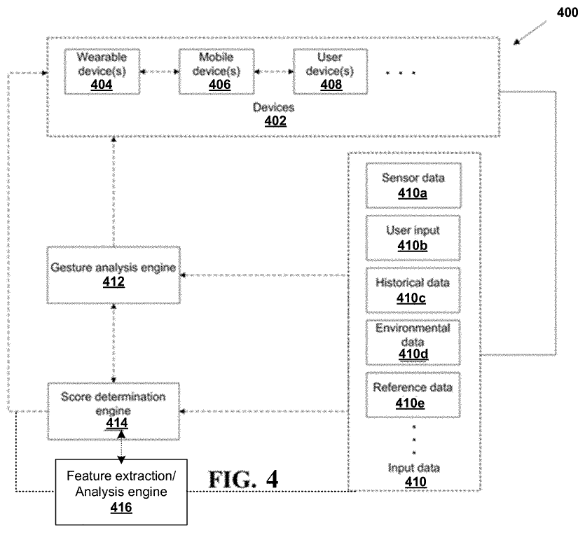

FIG. 4 illustrates various components in an example system in accordance with some embodiments. Referring to FIG. 4, an example system 400 may comprise one or more devices 402 such as a wearable device 404, a mobile device 406, and a user device 408, and one or more engines including such as gesture/event detection/analysis engine 412, score determination engine 414, feature extraction/analysis engine 416. The devices and engines may be configured to provide input data 410 including sensor data 410a, user input 410b, historical data 410c, environmental data 410d and reference data 410e.

As described above and elsewhere herein, the engines may be implemented inside and/or outside of a server. For example, the feature analysis engine may be software and/or hardware components included with a server, or remote from the server. In some embodiments, the feature analysis engine (or one or more functions of the feature analysis engine) may be implemented on the devices 402 while the gesture/event detection/analysis engine may be implemented on the server.

The devices may comprise sensors. The sensor data may comprise raw data collected by the devices. The sensor data may be stored in memory located on one or more of the devices (e.g., the wearable device). In some embodiments, the sensor data may be stored in one or more databases. The databases may be located on the server, and/or one or more of the devices. Alternatively, the databases may be located remotely from the server, and/or one or more of the devices.

The user input may be provided by a user via the devices. The user input may be in response to queries provided by the engines. Examples of queries may include whether the user is currently experiencing conditions associated with a given physical, psychological and/or physical and psychological state, whether the determined physical, psychological and/or physical and psychological state is accurate, whether the user needs help, whether the user needs medical assistance etc. The user's responses to the queries may be used to supplement the sensor data and/or detected gestures/events to determine one or more scores or user's physical, psychological and/or physical and psychological state.

The data may comprise user location data. The user location may be determined by a location sensor (e.g., GPS receiver). The location sensor may be on one or more of the devices such as the user device and/or the wearable device. The location data may be used to monitor user's activities. The location data may be used to determine user's points-of-interest. In some cases, multiple location sensors may be used to determine user's current location more accurately.

The historical data may comprise data collected over a predetermined time period. The historical data may be stored in memory located on the devices, and/or server. In some embodiments, the historical data may be stored in one or more databases. The databases may be located on the server, and/or the devices. Alternatively, the databases may be located remotely from the server, and/or the devices.

The environmental data may comprise data associated with an environment surrounding the user. The environmental data may comprise locations, ambient temperatures, humidity, sound level, or level of brightness/darkness of the environment where the user is located.

The feature analysis engine may be configured to analyze the sensor data and/or detected gestures/events to extract a plurality of features. In some embodiments, the feature analysis engine may be configured to calculate a multi-dimensional distribution function that is a probability function of a plurality of features in the sensor data and/or the detected gestures/events. The plurality of features may comprise n number of features denoted by p₁ through pₙ, where n may be any integer greater than 1. The multi-dimensional distribution function may be denoted by f(p₁, p₂, . . . , pₙ).

At least a portion of the plurality of features may be associated with various characteristics of a given physical, psychological or physical and psychological state. For example, in some embodiments, the plurality of features may comprise two or more of the following features: taking medication, drinking, falling, number of steps, sleeping (time, duration and/or quality of sleep), location(s). Accordingly, the multi-dimensional distribution function may be associated with one or more characteristics of a known physical, psychological or physical and psychological state, depending on the type of features that are selected and processed by the feature analysis engine. The multi-dimensional distribution function may be configured to return a single probability value between 0 and 1, with the probability value representing a probability across a range of possible values for each feature. Each feature may be represented by a discrete value. Additionally, each feature may be measurable along a continuum. The plurality of features may be encoded within the sensor data and/or the gestures/events, and extracted from the devices, gesture/event analysis engine and/or databases using the feature analysis engine.

In some embodiments, two or more features may be correlated. The feature analysis engine may be configured to calculate the multi-dimensional distribution function by using Singular Value Decomposition (SVD) to de-correlate the features such that they are approximately orthogonal to each other. The use of SVD can reduce a processing time required to compute a probability value for the multi-dimensional distribution function, and can reduce the amount of data required by the feature analysis engine to determine a high probability (statistically significant) that the user is at a given physical, psychological and/or physical and psychological state.

In some embodiments, the feature analysis engine may be configured to calculate the multi-dimensional distribution function by multiplying the de-correlated (rotated) 1D probably density distribution of each feature, such that the multi-dimensional distribution function f(p₁, p₂, . . . , pₙ)=f(p₁)*f(p₂)* . . . *f(pₙ). The function f(p₁) may be a 1D probability density distribution of a first feature, the function f(p₂) may be a 1D probability density distribution of a second feature, the function f(p₃) may be a 1D probability density distribution of a third feature, and the function f(pₙ) may be a 1D probability density distribution of a n-th feature. The 1D probability density distribution of each feature may be obtained from a sample size of each feature. In some embodiments, the sample size may be constant across all of the features. In other embodiments, the sample size may be variable between different features.

In some embodiments, the feature analysis engine may be configured to determine whether one or more of the plurality of features are statistically insignificant. For example, one or more statistically insignificant features may have a low correlation with a given physical, psychological and/or physical and psychological state. In some embodiments, the feature analysis engine may be further configured to remove the one or more statistically insignificant features from the multi-dimensional distribution function. By removing the one or more statistically insignificant features from the multi-dimensional distribution function, a computing time and/or power required to calculate a probability value for the multi-dimensional distribution function can be reduced.

### Examples

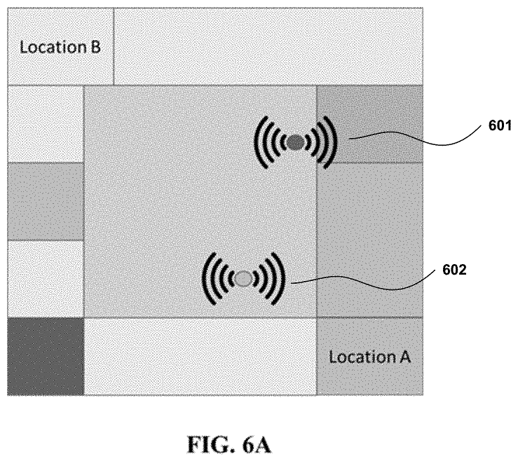

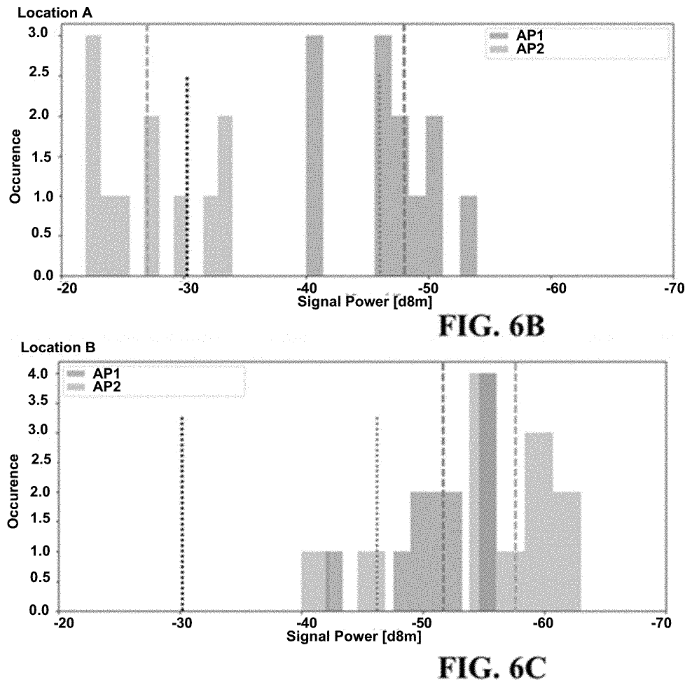

FIG. 6A shows a schematic of an example method for acquiring location data using location sensors. As shown in FIG. 6A, the outer square depicts the testing area. Two reference locations (i.e., location A and location B) are included in the testing area. Two wireless access points (AP's 601 and 602) (indicated by wifi-signals) are utilized to determine various locations in the testing area. For an unknown location, a distance between the average signal strength of each reference location and a signal from the unknown location may be calculated. The reference location which has the shortest distance from the unknown location may be selected as the location of a user. By using multiple AP's (e.g., greater than or equal to 3, 4, 5, 6, 7, 8, 9, 10, 12, 14, 16, 18, 20 or more), a location of the user can be easily distinguished.

Example testing results using the method of FIG. 6A are shown in FIGS. 6B and 6C. FIG. 6B shows signal strength distributions of two AP signals and a new signal obtained from location A over 1-hour time period. FIG. 6C shows signal strength distributions of two AP signals and a new signal obtained from location B over 1-hour time period. The AP1 has a similar distance from both reference locations, whereas the AP2 differs significantly. As shown in the figures, the new signal (dotted line) is closer to location A than location B, and thus location A is identified as the location of the user.

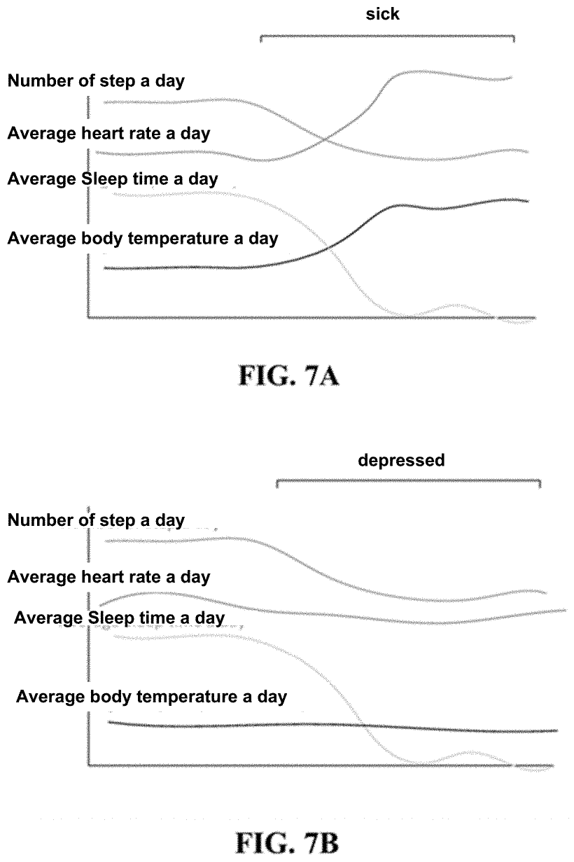

FIGS. 7A and 7B illustrate example sensor data, gestures and/or events associated with a physical state and psychological state, respectively. The sensor data, gestures and/or events comprise number of steps a day, average heart rate a day, average sleep time a day and average body temperature a day. The sensor data, gestures and/or events are collected or monitored over a pre-defined time period (e.g., ranging from hours, days to months or years). As described above or elsewhere herein, the same set of sensor data, gestures and/or events may be used for determining both the physical state and the psychological state. In some cases, however, different sets of sensor data, gestures and/or events may be used for determining the physical state and the psychological state respectively. The different sets of sensor data, gestures and/or events used for determining the physical state and the psychological state may comprise common sensor data, gestures and/or events. In some cases, the determination is based at least partially on the common sensor data, gestures and/or events.

Referring to FIGS. 7A and 7B, for both states, data curves of the number of steps a day and average sleep time a day may appear the same. However, data curves showing the average heart rate and body temperature differ significantly. For one state, both the average heart and body temperature increase over time, whereas no such increases can be observed for the other state. Depending at least partially on the data curves, a preliminary determination may be made. The data curves comprising relatively stable heart rate and body temperature may be determined to correspond to a depressed state, while the data curves showing elevated heart rate and body temperature may be determined to correspond to a sick state. The depressed state may be a state which may be indicative of or associated with depressive disorders of a subject. Depression may comprise persistent depressive disorder, postpartum depression, psychotic depression, seasonal affective disorder, or bipolar depression. Upon the determination, a notification including the data curves, results of the preliminary determination and/or queries to the user being monitored may be sent to the user and/or any other people or entities as described above or elsewhere herein. The user may provide responses to the queries. The responses may be used to supplement the sensor data and/or detected gestures/events to determine one or more scores or user's physical, psychological and/or physical and psychological state. For example, for the user who has been determined to have a depressed state, the queries may be sent to have the user to confirm whether he/she is experiencing a depressive disorder, whether the user have been diagnosed as having or likely to have a depressive disorder, whether the user have been taking medications for treating a depressive disorder, whether he/she needs medical assistance or help etc. Upon receipt or non-receipt of the user's responses to the queries, the set of sensor data, gestures and/or events may be adjusted, and/or the preliminary determination results may be updated.

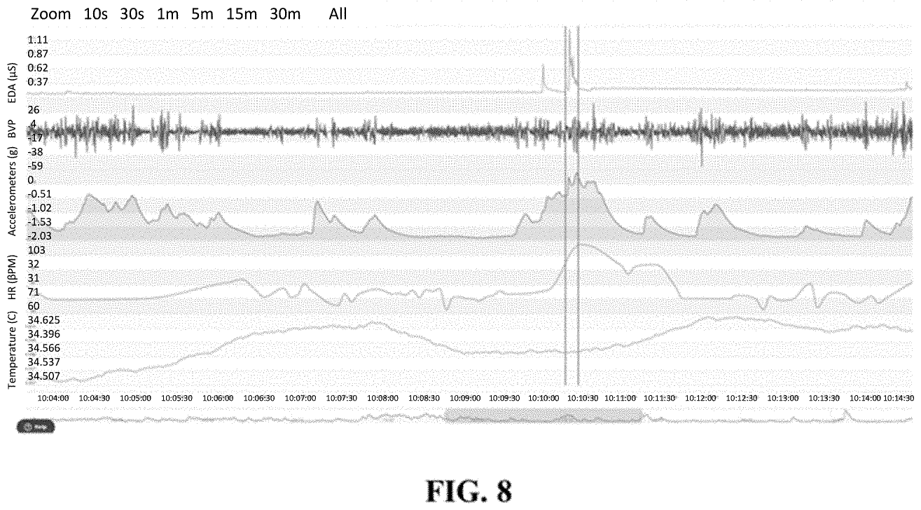

FIG. 8 shows example data collected over a per-defined time period which may be used for determining a physical score, a psychological score and/or a physical and psychological score. The data may comprise heart rate, body temperature, accelerometers, blood volume pulse (BVP) and electrodermal activity (EDA). Gestures and/or events may be detected using the data. One or more features may be extracted by analyzing or processing the data and/or gestures/events. Based on at least a subset of the features, one or more of a physical score, a psychological score and a total score may be determined. The scores may be indicative of or used to further determine a physical state, a psychological state and/or a physical and psychological state of a user.

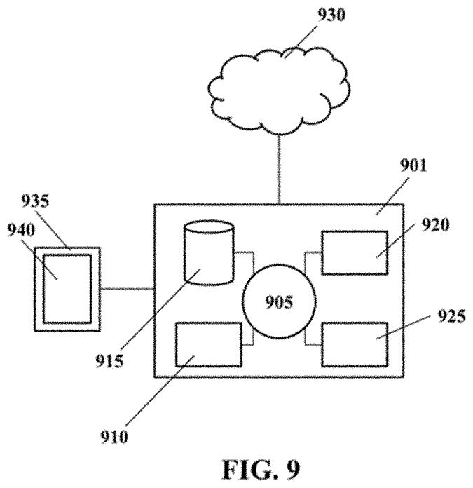

The present disclosure provides computer systems that are programmed to implement methods of the disclosure. FIG. 9 shows a computer system 901 that is programmed or otherwise configured to aggregate data associated with or collected using one or more devices, wherein the devices are configured to be carried or worn by the user; analyze the data to extract a plurality of features; and determine, based on one or more of the plurality of features, a user's well-being. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet.

The network 930 in some cases is a telecommunication and/or data network. The network 930 can include one or more computer servers, which can enable distributed computing, such as cloud computing. For example, one or more computer servers may enable cloud computing over the network 930 (“the cloud”) to perform various aspects of analysis, calculation, and generation of the present disclosure, such as, for example, detecting gestures and/or events, extracting features from device data and detected gestures/events, determining physical, psychological and/or total health scores, and generating a notification or result upon the determination. Such cloud computing may be provided by cloud computing platforms such as, for example, Amazon Web Services (AWS), Microsoft Azure, Google Cloud Platform, and IBM cloud. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user (e.g., a participant of a health incentive program). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc., shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, notification of detection of an abnormal event, a score indicating a progress has been made in a health program and the like. Examples of UI's include, without limitation, a graphical user interface (GUI) and web-based user interface.

As used herein A and/or B encompasses one or more of A or B, and combinations thereof such as A and B. It will be understood that although the terms “first,” “second,” “third” etc. may be used herein to describe various elements, components, regions and/or sections, these elements, components, regions and/or sections should not be limited by these terms. These terms are merely used to distinguish one element, component, region or section from another element, component, region or section. Thus, a first element, component, region or section discussed below could be termed a second element, component, region or section without departing from the teachings of the present disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A computer-implemented method for determining a user's well-being, the method comprising:
receiving data from a plurality of sources;
analyzing the data received from the plurality of sources to detect gestures and events associated with the user;
extracting a plurality of features from the data received from the plurality of sources and the analyzed data corresponding to the detected gestures and events;
selecting one or more subsets of features from the plurality of extracted features; and using at least partially the selected one or more subsets of features to determine (i) a physical state of the user, (ii) a psychological state of the user, or (iii) physical and psychological states of the user, to thereby determine the user's well-being.

2. The method of claim 1, wherein the one or more subsets of features comprise a first subset of features and a second subset of features, wherein the method further comprises using at least partially the first subset of features and the second subset of features to determine the physical state of the user and the psychological state of the user respectively.

3. (canceled)

4. The method of claim 1, wherein the one or more subsets of features comprise common feature, wherein the method further comprises using at least partially the common features to determine the physical and psychological state of the user.

5. (canceled)

6. The method of claim 1, further comprising: adjusting the one or more subsets of features if an accuracy of the determination is lower than a predetermined threshold.

7. The method of claim 4, wherein the accuracy is determined based on the user's feedback regarding the physical state, the psychological state, or the physical and psychological state of the user.

8. The method of claim 4, wherein the adjusting is performed by adding, deleting, or substituting one or more features in the one or more subsets of features.

9. The method of claim 4, wherein the adjusting is performed substantially in real-time.

10. The method of claim 1, further comprising: determining (1) a physical score based on the physical state of the user, (2) a psychological score based on the psychological state of the user, and/or (3) a total score based on the physical and psychological states of the use.

11. The method of claim 8, further comprising:
sending queries regarding the determined physical state, the psychological state, and/or the physical and psychological states to the user;
receiving responses to the queries from the user; and
adjusting the physical score, the psychological score, and/or the total score based on the responses.

12. The method of claim 1, further comprising: monitoring at least one of the physical and psychological states of the user as the gestures and events associated with the user are occurring.

13. The method of claim 10, further comprising: determining a trend of at least one of the physical and psychological states of the user based on the monitoring.

14. The method of claim 11, further comprising: predicting at least one of a future physical state or a psychological state of the user based on the trend.

15. The method of claim 1, further comprising: determining different degrees of a given physical state or psychological state, or distinguishing between different types of physical and psychological states.

16. (canceled)

17. The method of claim 1, wherein the plurality of sources comprises a wearable device and a mobile device associated with the user, and wherein the data comprises sensor data collected using a plurality of sensors on the wearable device or the mobile device.

18. (canceled)

19. The method of claim 1, wherein the gestures comprise different types of gestures performed by an upper extremity of the user.

20. The method of claim 1, wherein the events comprise (i) different types of activities and (ii) occurrences of low activity or inactivity.

21. The method of claim 1, wherein the events comprise walking, drinking, taking medication falling, eating, and/or sleeping.

22. The method of claim 1, wherein the plurality of features are processed using at least a machine learning algorithm or a statistical model.

23. The method of claim 1, wherein the physical state comprises a likelihood that the user is physically experiencing conditions associated with the physical state, and the psychological state comprises a likelihood that the user is mentally or emotionally experiencing conditions associated with the psychological state.

24. The method of claim 19, further comprising:
comparing the likelihood(s) to one or more thresholds; and
generating one or more alerts to the user or another entity, depending on whether the likelihood(s) are less than, equal, or greater than the one or more thresholds.

25.-42. (canceled)
